(19) United States
(12) Patent Application Publication
(10) Pub. No.: US 2010/0317688 A1
(43) Pub. Date: Dec. 16, 2010

(54) 2-HETEROAROYLIMIDAZOL[1,2-a]PYRIDINE DERIVATIVES, PREPARATION AND THERAPEUTIC USE THEREOF

(75) Inventor: Jean-Francois Peyronel, Palaiseau (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/828,388

(22) Filed: Jul. 1, 2010

Related U.S. Application Data
(63) Continuation of application No. PCT/FR2008/001840, filed on Dec. 31, 2008.

(30) Foreign Application Priority Data
Jan. 2, 2008 (FR) 0800009

(51) Int. Cl. A61K 31/437; C07D 471/04; A61P 25/00; A61P 25/28; A61P 25/18; A61P 29/00; A61P 19/10; A61P 35/00; A61P 25/16; A61P 25/24

(52) U.S. Cl. 514/300; 546/121

(57) ABSTRACT

Compounds of formula (I):

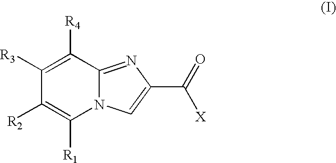

in which:
X, R₁, R₂, R₃, and R₄ are as defined in the disclosure, or an acid addition salt thereof; an therapeutic use thereof.

The present invention relates to 2-heteroaroylimidazo[1,2-a]pyridine derivatives, to their preparation and to their therapeutic use in the treatment or prevention of diseases involving the Nurr-1 nuclear receptors, also known as NR4A2, NOT, TINUR, RNR-1 and HZF3.

One subject of the present invention is compounds of formula (I):

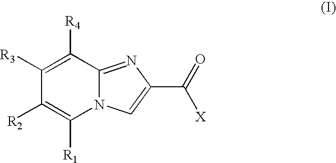

in which:
X represents a benzodioxole group, or a heteroaromatic group linked to the rest of the molecule via a carbon atom, this group being optionally substituted with one or more groups chosen, independently of each other, from a halogen atom and a group (C₁-C₆)alkyl, (C₁-C₆)alkoxy or NRaRb;
R₂ represents
   a hydrogen atom,
   a halogen atom,
   a group (C₁-C₆)alkyl optionally substituted with one or more atoms or groups chosen, independently of each other, from halogen, hydroxyl and NRaRb,
   a group (C₁-C₆)alkoxy optionally substituted with one or more atoms or groups chosen, independently of each other, from halogen, hydroxyl and NRaRb,
   a group (C₁-C₆)alkylthio,
   a group (C₂-C₆)alkenyl,
   a group (C₂-C₆)alkynyl,
   a group —CO—R₅,
   a group —CO—NR₆R₇,
   a group —CO—O—R₈,
   a group —NR₉—CO—R₁₀,
   a group —NR₁₁R₁₂,
   a cyano group,
   a phenyl group optionally substituted with one or more groups chosen, independently of each other, from halogen, (C₁-C₆)alkoxy, (C₁-C₆)alkyl optionally substituted with one or more atoms or groups: hydroxyl, NRcRd, CO—R₅, —CO—NR₆R₇, —CO—O—R₈, halogen or cyano,
   a heterocyclic group optionally substituted with one or more groups chosen, independently of each other, from the following atoms or groups: hydroxyl, halogen, (C₁-C₆)alkoxy, (C₁-C₆)alkyl optionally substituted with one or more hydroxyl, NRcRd, —CO—R₅, —CO—NR₆R₇, —CO—O—R₈, —NR₉—CO—R₁₀, cyano, an oxido group;
R₁ represents a hydrogen atom, a halogen atom, a group (C₁-C₆)alkyl, a group (C₁-C₆)alkoxy or a hydroxyl or amino group; the group (C₁-C₆)alkyl possibly being substituted with one or more of the atoms or groups: halogens, hydroxyl; amino, (C₁-C₆)alkoxy, and the group (C₁-C₆)alkoxy possibly being substituted with one or more of the atoms or groups: halogen, hydroxyl, amino, (C₁-C₆)alkoxy;
R₃ represents a hydrogen atom, a halogen atom or a group (C₁-C₆)alkyl or hydroxyl;
R₄ represents a hydrogen atom or a halogen atom;
R₅ represents a hydrogen atom or a group (C₁-C₆)alkyl;
R₆ and R₇, which may be identical or different, represent a hydrogen atom or a group (C₁-C₆)alkyl or form, with the nitrogen atom that bears them, a 4- to 7-membered ring optionally including another heteroatom chosen from N, O and S;
R₈ represents a group (C₁-C₆)alkyl;
R₉ and R₁₀, which may be identical or different, represent a hydrogen atom or a group (C₁-C₆)alkyl;
R₁₁ represents a group (C₁-C₆)alkyl;
R₁₂ represents a hydrogen atom or a group (C₁-C₆)alkyl;
   Ra and Rb represent, independently of each other, a hydrogen atom or a group (C₁-C₆)alkyl or form, with the nitrogen atom that bears them, a 4- to 7-membered ring, optionally including another heteroatom chosen from N, O and S;
   Rc and Rd represent a hydrogen atom or a (C₁-C₆)alkyl;
   with the exception of (5-bromo-2-benzofuranyl)imidazo[1,2-a]pyridin-2-ylmethanone and (5-methoxy-2-benzofuranyl)imidazo[1,2-a]pyridin-2-ylmethanone;
   in the form of the base or of an acid-addition salt.

The compounds of formula (I) may comprise one or more asymmetric carbon atoms. They may thus exist in the form of enantiomers or diastereoisomers. These enantiomers and diastereoisomers, and also mixtures thereof, including racemic mixtures, form part of the invention.

The compounds of formula (I) may exist in the form of bases or of acid-addition salts. Such addition salts form part of the invention.

These salts may be prepared with pharmaceutically acceptable acids, but the salts of other acids that are useful, for example, for purifying or isolating the compounds of formula (I) also form part of the invention.

The compounds of formula (I) may also exist in the form of hydrates or solvates, i.e. in the form of associations or combinations with one or more water molecules or with a solvent. Such hydrates and solvates also form part of the invention.

The compounds (5-bromo-2-benzofuranyl)imidazo[1,2-a]pyridin-2-ylmethanone and (5-methoxy-2-benzofuranyl)imidazo[1,2-a]pyridin-2-ylmethanone are respectively cited in chemical libraries under the numbers RN=382640-89-3 and RN=382612-77-3. No pharmaceutical or therapeutic activity has been demonstrated for these compounds. They have been specifically excluded from formula (I) according to the invention.

A method for identifying compounds that inhibit cdc 34 is moreover known from document US 2006/0 211 747, one of the identified compounds being an imidazo[1,2-a]pyridine, which is not included in formula (I) according to the present invention.

Benzoyl-2-imidazo[1,2-a]pyridine derivatives, which are useful as medicaments, are also known from FR 2 638 161.

In the context of the present invention, the following definitions apply:
a halogen atom: a fluorine, a chlorine, a bromine or an iodine;
an alkyl group: a linear, branched or cyclic, saturated aliphatic group, optionally substituted with a linear, branched or cyclic, saturated alkyl group. Examples that may be mentioned include methyl, ethyl, propyl, isopropyl, butyl, isobutyl, tert-butyl, cyclopropyl, cyclobutyl, cyclopentyl, cyclohexyl, methylcyclopropyl, etc. groups;
a group (C₂-C₆)alkenyl: a linear or branched, mono- or polyunsaturated aliphatic group of 2 to 6 carbons, comprising, for example, one or two ethylenic unsaturations;
a group (C₁-C₆)alkoxy: a radical —O-alkyl in which the alkyl group is as defined previously;
a group (C₂-C₆)alkynyl: a linear or branched, mono- or polyunsaturated aliphatic group of 2 to 6 carbons, comprising, for example, one or two ethylenic unsaturations;
a heteroaromatic group: a monocyclic or bicyclic, unsaturated or partially unsaturated group, comprising from 5 to 10 atoms including 1 to 4 heteroatoms chosen from N, O and S. Examples of heteroaromatic groups that may be mentioned include: pyrrole, furan, thiophene, pyrazole, imidazole, triazole, tetrazole, oxazole, isoxazole, oxadiazole, thiazole, isothiazole, thiadiazole, pyridine, pyrimidine, pyrazine, pyridazine, triazine, pyrrolopyrrole, pyrroloimidazole, pyrrolopyrazole, pyrrolotriazole, imidazoimidazole, imidazopyrazole, imidazotriazole, indole, isoindole, benzimidazole, indazole, indolizine, benzofuran, isobenzofuran, benzothiophene, benzo[c]thiophene, pyrrolopyridine, imidazopyridine, pyrazolopyridine, triazolopyridine, tetrazolopyridine, pyrrolopyrimidine, imidazopyrimidine, pyrazolopyrimidine, triazolopyrimidine, tetrazolopyrimidine, pyrrolopyrazine, imidazopyrazine, pyrazolopyrazine, triazolopyrazine, tetrazolopyrazine, pyrrolopyridazine, imidazopyridazine, pyrazolopyridazine, triazolopyridazine, tetrazolopyridazine, pyrrolotriazine, imidazotriazine, pyrazolotriazine, triazolotriazine, tetrazolotriazine, furopyridine, furopyrimidine, furopyrazine, furopyridazine, furotriazine, oxazolopyridine, oxazolopyrimidine, oxazolopyrazine, oxazolopyridazine, oxazolotriazine, isoxazolopyridine, isoxazolopyrimidine, isoxazolopyrazine, isoxazolopyridazine, isoxazolotriazine, oxadiazolopyridine, oxadiazolopyrimidine, oxadiazolopyrazine, oxadiazolopyridazine, oxadiazolotriazine, benzoxazole, benzisoxazole, benzoxadiazole, thienopyridine, thienopyrimidine, thienopyrazine, thienopyridazine, thienotriazine, thiazolopyridine, thiazolopyrimidine, thiazolopyrazine, thiazolopyridazine, thiazolotriazine, isothiazolopyridine, isothiazolopyrimidine, isothiazolopyrazine, isothiazolopyridazine, isothiazolotriazine, thiadiazolopyridine, thiadiazolopyrimidine, thiadiazolopyrazine, thiadiazolopyridazine, thiadiazolotriazine, benzothiazole, benzoisothiazole, benzothiadiazole, quinoline, isoquinoline, cinnoline, phthalazine, quinoxaline, quinazoline, naphthyridine, benzotriazine, pyridopyrimidine, pyridopyrazine, pyridopyridazine, pyridotriazine, pyrimidopyrimidine, pyrimidopyrazine, pyrimidopyridazine, pyrimidotriazine, pyrazinopyrazine, pyrazinopyridazine, pyrazinotriazine, pyridazinopyridazine, pyridazinotriazine.
a heterocyclic group: a heteroaromatic group as defined above, which may also optionally be saturated.

It should be noted that, in the context of the present invention, the envisaged radicals may be named, without preference, by addition or otherwise of the suffix “-yl”. For example, “benzodioxole” is the same as “benzodioxolyl”.

Various subgroups of compounds are defined hereinbelow, and also form part of the invention.

Among the compounds of general formula (I) that are subjects of the invention as defined previously, a second group of compounds is constituted by the compounds for which at least one from among R₁, R₂, R₃ and R₄ is other than a hydrogen atom, the other groups being as defined previously.

Among the compounds of general formula (I) that are subjects of the invention as defined previously, a first group of compounds is constituted by compounds for which at least one from among R₁ and R₂ is other than a hydrogen atom, the other groups being as defined previously.

Among the compounds of general formula (I) that are subjects of the invention as defined previously, a third group of compounds is constituted by compounds for which R₂ represents one of the following groups:
a hydrogen atom,
a halogen atom,
a group (C₁-C₆)alkyl,
a monocyclic heterocyclic group of 5 or 6 atoms, optionally substituted with one or more groups chosen, independently of each other, from the following atoms or groups: hydroxyl, halogen, (C₁-C₆)alkoxy, (C₁-C₆)alkyl optionally substituted with one or more hydroxyl, NRcRD, —CO—R₅, —CO—NR₆R₇, —CO—O—R₈, —NR₉—CO—R₁₀, cyano, an oxido group;

R₅ represents a hydrogen atom or a group (C₁-C₆)alkyl;

Rc and Rd represent a hydrogen atom;

the other groups being as defined previously.

Among the compounds of general formula (I) that are subjects of the invention as defined previously, a fourth group of compounds is constituted by compounds for which R₂ represents one of the following groups:
a hydrogen atom,
a halogen atom,
a group (C₁-C₆)alkyl,
a pyridine group,

the other groups being as defined previously.

Among the compounds of general formula (I) that are subjects of the invention as defined previously, a fifth group of compounds is constituted by compounds for which X represents a pyridine, benzoxazole, thiophene, furan, benzodioxole or benzothiazole group, the other groups being as defined previously.

Among the compounds of general formula (I) that are subjects of the invention as defined previously, a sixth group of compounds is constituted by compounds for which:

R₁ represents a hydrogen atom or a group (C₁-C₆)alkyl,

R₃ and R₄ represent a hydrogen atom;

R₂ represents a hydrogen atom, a chlorine atom, a methyl group or a pyridine group;

X represents a benzoxazole, thiophene, furan, benzodioxole or benzothiazole group, the said groups being linked to the rest of the molecule via a carbon atom;

and at least one from among R₁ and R₂ is not hydrogen,
in the form of the base or of an acid-addition salt.

Among the compounds of formula (I) that are subjects of the invention, a seventh group of compounds is constituted of compounds for which:

R₁ represents a hydrogen atom or a methyl group,

R₃ and R₄ represent a hydrogen atom;

R₂ represents a hydrogen atom, a chlorine atom, a methyl group or a pyridine group;

X represents a pyridine, benzoxazole, thiophene, furan, benzodioxole or benzothiazole group, and at least one from among R₁, R₂, R₃ and R₄ is not hydrogen, in the form of the base or of an acid-addition salt.

Among the compounds of formula (I) that are subjects of the invention, mention may be made especially of the following compounds:
(6-Chloroimidazo[1,2-a]pyridin-2-yl)(pyridin-2-yl)methanone
Benzoxazol-2-yl(6-chloroimidazo[1,2-a]pyridin-2-yl)methanone
(6-Chloroimidazo[1,2-a]pyridin-2-yl)(3-furyl)methanone
(6-Chloroimidazo[1,2-a]pyridin-2-yl)(thien-2-yl)methanone
(6-Chloroimidazo[1,2-a]pyridin-2-yl)(thien-3-yl)methanone
1,3-Benzodioxol-5-yl(6-chloroimidazo[1,2-a]pyridin-2-yl)methanone
Benzothiazol-2-yl(6-chloroimidazo[1,2-a]pyridin-2-yl)methanone
(6-Methylimidazo[1,2-a]pyridin-2-yl)(thien-2-yl)methanone
(5-Methylimidazo[1,2-a]pyridin-2-yl)(thien-2-yl)methanone
(6-Pyridin-2-yl)imidazo[1,2-a]pyridin-2-yl)(thien-2-yl)methanone

In accordance with the invention, the compounds of general formula (I) may be prepared according to the process described in Scheme 1.

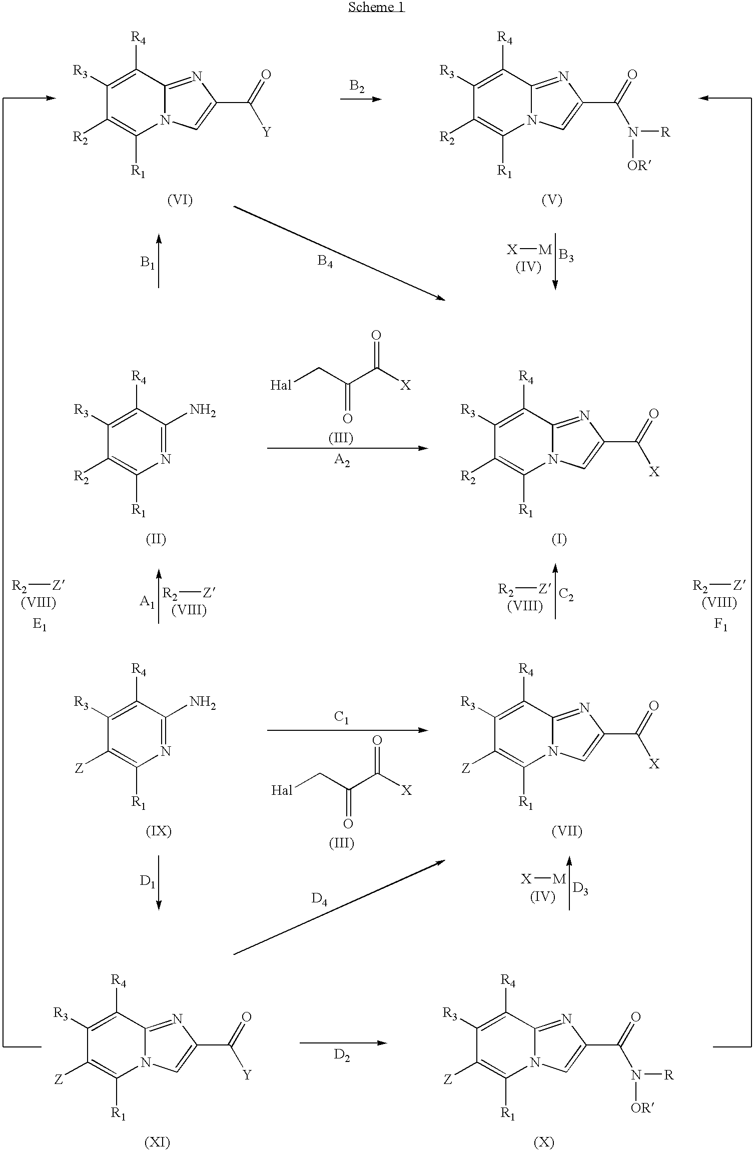

The first synthetic route (transformation A₂) consists in condensing a 2-aminopyridine of formula (II), in which R₁, R₂, R₃ and R₄ are as defined previously, with a 3-halo-1-(hetero)arylpropane-1,2-dione derivative of general formula (III), in which Hal represents a chlorine, bromine or iodine atom and X is as defined previously, to form the imidazo[1,2-a]pyridine ring, for example according to the method described by J-J. Bourguignon et al. in Aust. J. Chem., 50, 719 (1997).

The second synthetic route (transformation B₃ or B₄) consists in reacting an organometallic derivative of general formula (IV), in which X is as defined previously and M represents a lithium atom or a group Mg-Hal:
with a Weinreb amide (N-alkoxy-N-alkylamide) of general formula (V), in which R₁, R₂, R₃ and R₄ are as defined previously and are other than bromine or iodine and R and R′, which may be identical or different, represent an alkyl group, according to methods known to those skilled in the art, as described by Weinreb, S. M. et al. in Tetrahedron Letters (1981), 22(39), 3815-18 and in Sibi, M. P. Organic Preparations and Procedures Int. 1993, 25, 15-40 (transformation B₃), or
with an imidazo[1,2-a]pyridine-2-carboxylic acid of general formula (VI), in which R₁, R₂, R₃ and R₄ are as defined previously and are other than bromine or iodine and Y represents a hydroxyl group or a salt or reactive derivative thereof such as an ester, acid halide, anhydride or amide, according to methods known to those skilled in the art, as described in J. March, Advanced Organic Chemistry (Wiley, 5th Ed. 2001) pp. 567 and 1213 or in the cited references (transformation B₄).

Transformation B₄ may also be performed by reacting a reactive derivative such as a mixed anhydride (which may be generated in situ) of the imidazo[1,2-a]pyridine-2-carboxylic acid of formula (VI), in which Y represents a hydroxyl group and R₁, R₂, R₃ and R₄ are as defined previously and are other than bromine or iodine, with an organometallic derivative of formula (IV), in which X is defined as above and M represents a boronic group, in the presence of a palladium catalyst such as tetrakis(triphenylphosphine)palladium.

The third synthetic route (transformation C₂) consists in performing the catalytic coupling of a derivative of general formula (VII), in which R₁, R₃ and R₄ are as defined previously and Z represents a boryl, stannyl or silyl group, with a derivative R₂-Z′ (VIII), in which Z′ represents a halogen atom such as bromine or iodine or a sulfonyloxy group and R₂ is an optionally substituted 1-alkenyl, 1-alkynyl, aryl or heteroaryl group. Alternatively, the coupling may be performed between a derivative of general formula (VII) in which R₁, R₃ and R₄ are as defined previously and Z represents a halogen atom such as bromine or iodine, with a derivative R₂-Z′ (VIII), in which Z′ represents a reactive group such as a boryl, stannyl or silyl group or a hydrogen atom and R₂ is an optionally substituted 1-alkenyl, 1-alkynyl, aryl or heteroaryl group.

The 2-aminopyridines of formula (II) may be prepared according to the methods described in the literature or known to those skilled in the art. In particular, the 2-aminopyridines of formula (II), in which R₁, R₃ and R₄ are as defined previously and R₂ is an optionally substituted 1-alkenyl, 1-alkynyl, aryl or heteroaryl group, may be prepared via transformation A₁, i.e. via a catalytic coupling reaction,
either of a 2-aminopyridine derivative of formula (IX) in which R₁, R₃ and R₄ are as defined previously and Z represents a boryl, stannyl or silyl group, with a derivative R₂-Z′ (VIII), in which Z′ represents a halogen atom such as bromine or iodine or a sulfonyloxy group and R₂ is an optionally substituted 1-alkenyl, 1-alkynyl, aryl or heteroaryl group,
or of a 2-aminopyridine derivative of formula (IX), in which R₁, R₃ and R₄ are as defined previously and Z represents a halogen atom such as bromine or iodine, with a derivative R₂-Z′ (VIII) in which Z′ represents a reactive group such as a boryl, stannyl or silyl group, or a hydrogen atom and R₂ is an optionally substituted 1-alkenyl, 1-alkynyl, aryl or heteroaryl group.

The 3-halo-1-(hetero)arylpropane-1,2-dione derivatives of formula (III) may be prepared by halogenation of the corresponding 1-(hetero)arylpropane-1,2-diones according to the methods known to those skilled in the art.

The Weinreb amides of formula (V) may be obtained (transformation B₂) by coupling an acid of formula (VI), in which Y represents a hydroxyl group or a reactive derivative thereof, with an N,O-dialkylamine according to the methods known to those skilled in the art. The coupling may be performed in the presence of a coupling agent such as CDI, EDCI, HATU or HBTU and of a base such as diisopropylethylamine, triethylamine or pyridine, in an inert solvent such as THF, DMF or dichloromethane. Alternatively, the N,O-dialkylamine may be reacted with an ester of formula (VI) in which Y represents an alkoxy group, in the presence of a catalyst such as trimethylaluminium (Weinreb. S. M. et al., Synth. Commun 1982, 12, 989).

The imidazopyridine-2-carboxylic acid derivatives of formula (VI) in which R₁, R₂, R₃ and R₄ are as defined previously and Y is (C₁-C₆)alkoxy, hydroxy or halogen may be prepared by condensation of a 2-aminopyridine of formula (II), in which R₁, R₂, R₃ and R₄ are as defined previously, with a 3-halo-2-oxopropionic acid ester of formula (VIII) in which Hal represents a chlorine, bromine or iodine atom and Y is (C₁-C₆)alkoxy, under the conditions described by J. G. Lombardino in J. Org. Chem., 30, 2403 (1965), for example, followed, where appropriate, by conversion of the ester into the acid and then into the acid chloride or another reactive derivative (transformation B₁).

The imidazo[1,2-a]pyridine derivatives of formula (VII), in which X, R₁, R₃ and R₄ are as defined previously and Z represents a halogen atom or a boryl, stannyl or silyl group, may be prepared (transformation C₁) by condensation of a 2-aminopyridine of formula (II) in which Z, R₁, R₃ and R₄ are as defined above, with a 3-halo-1-(hetero)arylpropane-1,2-dione derivative of general formula (III), in which Hal represents a chlorine, bromine or iodine atom, under the conditions described above the preparation of the products of general formula (I), via transformation A₂.

Alternatively, the imidazo[1,2-a]pyridine derivatives of formula (VII), in which X, R₁, R₃ and R₄ are as defined previously and Z represents a halogen atom or a boryl, stannyl or silyl group, may be prepared by reaction of an organometallic derivative of general formula (IV), in which X is as defined previously and M represents a lithium atom or a group Mg-Hal, with an imidazo[1,2-a]pyridine-2-carboxylic acid of formula (XI), in which R₁, R₂, R₃, R₄ and Z are as defined above and are other than bromine or iodine and Y represents a hydroxyl group, or a reactive derivative thereof such as an acid chloride (transformation D₄) or a corresponding Weinreb amide of formula (X) (transformation D₃), while optionally protecting the other reactive functions under the conditions described above for the preparation of the products of general formula (I), via transformation B₃ or B₄.

The imidazopyridine-2-carboxylic acid derivatives of formulae (X) and (XI) may be prepared by condensation of a 2-aminopyridine of formula (IX), in which Z, R₁, R₃ and R₄ are as defined previously, with a 3-halo-2-oxopropionic acid ester of formula (VIII), in which Hal represents a chlorine, bromine or iodine atom and Y is (C₁-C₆)alkoxy, according to the methods described above for the preparation of the derivatives of formulae (V) and (VI) (transformation D₁).

The coupling of the derivatives of formula (VII), (IX) or (X) with the products of formula (VIII) may be performed via any method known to those skilled in the art, in particular working in the presence of copper-based or palladium-based catalysts or ligands such as phosphines, according to or by analogy with the methods described, for example, in the following references and cited references:

for the reactions of Suzuki type: N. Miyaura, A. Suzuki, Chem. Rev., 95, 2457, (1995),

for the reactions of Stille type: V. Farina et al., Org. React., 50, 1 (1997),

for the reactions of Hiyama type: T. Hiyama et al., Top. Curr. Chem., 2002, 219, 61 (2002),

for the reactions of Negishi type: E. Negishi et al., Chem. Rev., 103, 1979 (2003),

for the reactions of Bellina type: M. Miura et al., Chem. Lett., 200 (2007).

It is also possible, in order to perform the coupling, to form as intermediates, but without isolating them, organometallic derivatives such as zinc derivatives.

In accordance with the invention, the compounds of general formulae (I), (II) and (VI) may also be prepared according to the processes described in Scheme 2, i.e. via conversion of a compound of general formula (XII), (XIII) or (XIV), in which R₁, R₃, R₄ and X are as defined previously, Y is a hydroxyl, alkoxy or N-alkoxy-N-alkylamino group and W represents a precursor group for constructing the heterocycle of formula R₂, respectively, as compounds of general formulae (I), (VI) and (II), according to the methods known to those skilled in the art (transformations G₁, G₂ and G₃).

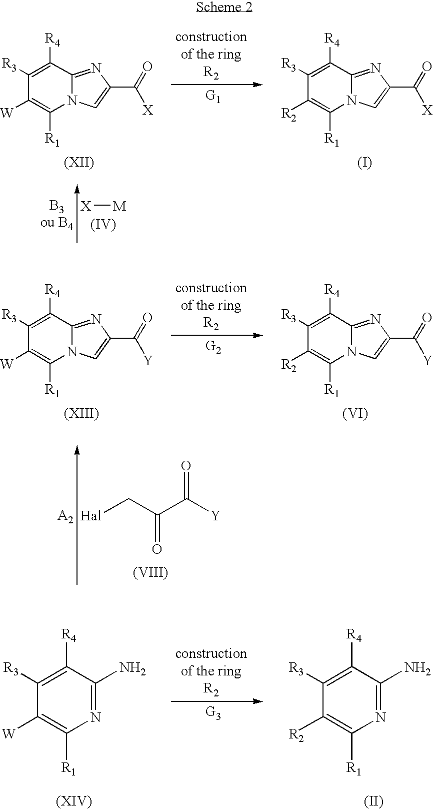

By way of example, W may represent:

a 2-haloacyl group such as bromoacetyl, or a 1-halo-2-oxoalkyl group such as 1-bromo-2-oxoethyl, which may be converted, for example, into a thiazolyl, imidazolyl or oxazolyl group by treatment with thiourea, thioamide, guanidine, urea or amide derivatives,

an alkynyl group, such as ethynyl, which may be converted into a 1,2,3-triazol-4-yl group,

a cyano group, which may be converted, for example, into a dihydroimidazolyl (2) or 1,3,4-triazol-2-yl group.

The compounds of general formula (XII) may be obtained from the compounds of formula (XIII) under the conditions described for the preparation of compounds (I) from the imidazopyridine-2-carboxylic acid derivatives of formula (V) or (VI), via transformation B₂ or B₄.

The imidazopyridine-2-carboxylic acid derivatives of general formula (XIII) may be obtained from the aminopyridines of formula (XIV) under the conditions described for the conversion of the aminopyridines of formula (II) into compounds of general formula (I), via transformation A₂.

The products of formula (I) and the precursors thereof of formula (II), (V) or (VI) may be subjected, if desired and if necessary, in order to obtain products of formula (I) or to be converted into other products of formula (I), to one or more of the following transformation reactions, in any order:
a) a reaction for the esterification or amidation of an acid function,
b) a reaction for the hydrolysis of an ester function to an acid function,
c) a reaction for the transformation of a hydroxyl function into an alkoxy function,
d) a reaction for the oxidation of an alcohol function to an aldehyde or ketone function,
e) a reaction for the oxidation of an alkenyl group to an aldehyde or ketone function,
f) a reaction for the dehydration of a hydroxyalkyl group to an alkenyl group,
g) a reaction for the total or partial hydrogenation of an alkenyl or alkynyl group to an alkenyl or alkyl group,
h) a catalytic coupling reaction of a halogenated derivative and of an organometallic derivative such as a tin or boron derivative to introduce an alkyl, alkenyl, alkynyl, aryl or heteroaryl substituent,
i) a reaction for the conversion of a halogenated derivative to introduce a boryl, stannyl or silyl substituent,
j) a reaction for the protection of reactive functions,
k) a reaction for the removal of the protecting groups that may be borne by the protected reactive functions,
l) a salification reaction with a mineral or organic acid or with a base, to obtain the corresponding salt,
m) a reaction for the resolution of racemic forms into enantiomers,
   the said products of formula (I) thus obtained being, where appropriate, in any possible isomeric form: racemic mixtures, enantiomers and diastereoisomers.

In Scheme 1, the starting compounds and the reagents, when their mode of preparation is not described, are commercially available or are described in the literature, or else may be prepared according to methods that are described therein or that are known to those skilled in the art.

The examples that follow describe the preparation of certain compounds in accordance with the invention. These examples are not limiting, but serve merely to illustrate the present invention. The numbers of the illustrated compounds refer to those given in the table hereinbelow, which illustrates the chemical structures and physical properties of a number of compounds according to the invention.

### EXAMPLE 1

### (6-Chloroimidazo[1,2-a]pyridin-2-yl)(pyridin-2-yl)methanone

To a solution of 1.03 g of 2-iodopyridine in 25 mL of tetrahydrofuran cooled to −20° C. are added dropwise 6 mL of a 1M solution of ethylmagnesium bromide in tert-butyl methyl ether. The reaction mixture is stirred for 20 minutes at −20° C. 0.24 g of N-methoxy-N-methyl-6-chloroimidazo[1,2-a]pyridine-2-carboxamide is added. The reaction mixture is stirred at −20° C. for 2 hours. The cooling bath is removed and stirring is continued for a further 2 hours. 10 mL of saturated ammonium chloride solution, 10 mL of water and 40 mL of ethyl acetate are added. After separation of the phases by settling, the organic phase is dried over magnesium sulfate and evaporated to dryness under reduced pressure. The residue is purified by chromatography on a column of silica, eluting with a 95/5 mixture of dichloromethane and methanol. The fractions containing the expected product are combined and concentrated to dryness under reduced pressure to give 0.153 g of (6-chloroimidazo[1,2-a]pyridin-2-yl)-pyridin-2-ylmethanone in the form of a white solid.

### EXAMPLE 2

### 1,3-Benzoxazol-2-yl(6-chloroimidazo[1,2-a]pyridin-2-yl)methanone

To a solution of 163 mg of 1,3-benzoxazole in THF at −78° C. is added 0.85 mL of a 1.6 M solution of n-butyllithium in hexane. After 30 minutes, 163 mg of N-methoxy-N-methylamide dissolved in THF are added and the reaction mixture is stirred at room temperature for 16 hours. Saturated aqueous ammonium chloride solution is added and the mixture is extracted with ethyl acetate. The organic phase is dried over magnesium sulfate and evaporated to dryness under reduced pressure. The residue is purified by chromatography on a column of silica, eluting with a 95/5 mixture of dichloromethane and methanol. The fractions containing the expected product are combined and concentrated to dryness under reduced pressure to give 36 mg of 1,3-benzoxazol-2-yl(6-chloroimidazo[1,2-a]pyridin-2-yl)methanone in the form of a white solid.

### EXAMPLE 3

### (6-Chloroimidazo[1,2-a]pyridin-2-yl)(3-furyl)methanone

To a solution of 98 mg of 6-chloroimidazo[1,2-a]pyridine-2-carboxylic acid in 2 mL of dioxane are added 288 mg of dimethyl dicarbonate, 23 mg of tetrakis(triphenylphosphine)palladium and 154 mg of furan-3-ylboronic acid. The reaction mixture is heated at 110° C. for 16 hours, saturated aqueous sodium bicarbonate solution is then added and the mixture is extracted with dichloromethane. The combined organic phases are dried over sodium sulfate and evaporated to dryness under reduced pressure. The residue is purified by chromatography on a column of silica, eluting with a 95/5 mixture of dichloromethane and methanol. The fractions containing the expected product are combined and concentrated to dryness under reduced pressure to give 23 mg of (6-chloroimidazo[1,2-a]pyridin-2-yl)(3-furyl)methanone in the form of a yellow solid.

### EXAMPLE 4

### (6-Chloroimidazo[1,2-a]pyridin-2-yl)(thien-2-yl)methanone

To a solution of 100 mg of N-methoxy-N-methyl-6-chloroimidazo[1,2-a]pyridine-2-carboxamide cooled to 0° C. are added dropwise 1.1 mL of a 1 M solution of thiophen-2-ylmagnesium bromide in THF. The reaction medium is stirred at room temperature for 16 hours. Saturated ammonium chloride solution is added and the mixture is extracted with ethyl acetate. The combined organic phases are dried over sodium sulfate and evaporated to dryness under reduced pressure. The residue is purified by chromatography on a column of silica, eluting with a 9/1 mixture of dichloromethane and ethyl acetate. The fractions containing the expected product are combined and concentrated to dryness under reduced pressure to give 65 mg of (6-chloroimidazo[1,2-a]pyridin-2-yl)(thien-2-yl)methanone in the form of a white solid.

The intermediates described below are useful for preparing the compounds of the present invention.

### Intermediate 1: N-methoxy-N-methyl-6-chloroimidazo[1,2-a]pyridine-2-carboxamide

To a solution of 0.784 g of 6-chloroimidazo[1,2-a]pyridine-2-carboxylic acid in 12 mL of dichloromethane are added 1.67 mL of triethylamine, 1.53 g of 1-(3-dimethylaminopropyl)-3-ethylcarbodiimide hydrochloride and 1.08 g of 1-hydroxybenzotriazole. The reaction mixture is stirred for 20 minutes at room temperature. 0.39 g of N—O-dimethylhydroxylamine is added. The reaction mixture is stirred for 4 hours at room temperature. 60 mL of dichloromethane and 30 mL of water are added. After separation of the phases by settling, the organic phase is dried over magnesium sulfate, filtered and evaporated to dryness under reduced pressure, and then purified on a column of silica, eluting with a 95/5 mixture by volume of dichloromethane and methanol. The fractions containing the product are combined and concentrated to dryness under reduced pressure to give 0.6 g of N-methoxy N-methyl 6-chloroimidazo[1,2-a]pyridine-2-carboxamide in the form of a white solid.

¹H NMR spectrum (DMSO-d6, δ in ppm): 3.42 (broad s, 3H); 3.75 (s, 3H); 7.37 (dd, J=2.0 and 9.5 Hz, 1H); 7.67 (d, J=9.5 Hz, 1H); 8.39 (s, 1H); 8.85 (d, J=2.0 Hz, 1H).

Mass spectrum (LCMS): m/z 240: [M+H]⁺.

### Intermediate 2: N-methoxy-N-methyl-5-methylimidazo[1,2-a]pyridine-2-carboxamide

N-Methoxy-N-methyl-5-methylimidazo[1,2-a]pyridine-2-carboxamide is prepared from 5-methylimidazo[1,2-a]pyridine-2-carboxylic acid under conditions similar to those described for the preparation of intermediate 1.

¹H NMR spectrum (DMSO-d6, δ in ppm): 2.64 (s, 3H); 3.47 (broad s, 3H); 3.77 (s, 3H); 6.85 (broad d, J=7.0 Hz, 1H); 7.30 (dd, J=7.0 and 9.0 Hz, 1H); 7.51 (broad d, J=9.0 Hz, 1H); 8.21 (s, 1H).

Mass spectrum (EI): m/z 219: [M+.], m/z 188: [M+.]-OCH3, m/z 159 (base peak): [M+.]-C2H6NO.

### Intermediate 3: N-methoxy-N-methyl-6-methylimidazo[1,2-a]pyridine-2-carboxamide

N-Methoxy-N-methyl-6-methylimidazo[1,2-a]pyridine-2-carboxamide is prepared from 6-methylimidazo[1,2-a]pyridine-2-carboxylic acid under conditions similar to those described for the preparation of intermediate 1.

Mass spectrum (EI): m/z 219: [M]⁺, m/z 188: [M−OCH₃]⁺, m/z 159 (base peak): [M−C₂H₆NO]⁺.

### Intermediate 4: N-methoxy-N-methyl-6-methylimidazo[1,2-a]pyridine-2-carboxamide

### 1. Ethyl 6-(4,4,5,5-tetramethyl-1,3,2-dioxaborolan-2-yl)imidazo[1,2-a]pyridine-2-carboxylate hydrobromide

To a solution of 4 g of 2-amino-5-(4,4,5,5-tetramethyl-1,3,2-dioxaborolan-2-yl)pyridine in 40 mL of 1,2-dimethoxyethane are added 4.26 g of ethyl 3-bromo-2-oxopropionate. The reaction mixture is stirred for 40 hours at 20° C. The precipitate is filtered off by suction, washed with a small amount of 1,2-dimethoxyethane and pentane and then taken up in 50 mL of ethanol and refluxed for 1 hour. The reaction mixture is concentrated to dryness under reduced pressure. The oil obtained is redissolved in ethyl ether and the solution is concentrated under reduced pressure. The solid is filtered off by suction and washed with a small amount of ethyl ether to give 3.78 g of ethyl 6-(4,4,5,5-tetramethyl-1,3,2-dioxaborolan-2-yl)imidazo[1,2-a]pyridine-2-carboxylate hydrobromide in the form of a white solid.

¹H NMR spectrum (DMSO-d6, δ in ppm): 1.27-1.38 (m, 15H), 4.36 (q, J=7.3 Hz, 2H), 7.59 (d, J=9.3 Hz, 1H), 7.67 (d, J=9.3 Hz, 1H), 8.68 (s, 1H), 8.97 (s, 1H).

Mass spectrum (EI): m/z 316 [M]⁺, 244 [M−CO₂Et+H]⁺.

### 2. Ethyl 6-pyridin-2-ylimidazo[1,2-a]pyridine-2-carboxylate

A mixture of 3.18 g of caesium carbonate, 25 mL of dioxane, 9.3 mL of water, 500 mg of 2-iodopyridine, 89 mg of [1,1′-bis(diphenylphosphino)ferrocene]dichloropalladium and 848 mg of ethyl 6-(4,4,5,5-tetramethyl-1,3,2-dioxaborolan-2-yl)imidazo[1,2-a]pyridine-2-carboxylate hydrobromide is heated for 2 hours at 110° C., and then partially concentrated and diluted with dichloromethane and filtered. The organic phase is washed with water and dried over magnesium sulfate, filtered and concentrated to dryness under reduced pressure. The residue is chromatographed on a silicone cartridge, eluting with a mixture of dichloromethane and cyclohexane (80/20). The fractions containing the expected product are combined and concentrated to dryness under reduced pressure to give 317 mg of ethyl 6-pyridin-2-ylimidazo[1,2-a]pyridine-2-carboxylate in the form of a brown oil.

¹H NMR spectrum (DMSO-d6, δ in ppm): 1.34 (t, J=7.0 Hz, 3H), 4.33 (q, J=7.0 Hz, 2H), 7.42 (ddd, J=7.5, 5.5, 2.0 Hz, 1H), 7.73 (d, J=9.3 Hz, 1H), 7.85-8.02 (m, 2H), 8.07 (dd, J=9.3, 2.0 Hz, 1H), 8.64 (s, 1H), 8.70 (broad d, J=5.5 Hz, 1H), 9.36 (broad s, 1H).

Mass spectrum (LC-MS-DAD-ELSD): m/z 268 [M+H]⁺.

### 3: 6-Pyridin-2-ylimidazo[1,2-a]pyridine-2-carboxylic acid

1.78 mL of aqueous 2 M lithium hydroxide solution are added to a solution of 317 mg of ethyl 6-pyridin-2-ylimidazo[1,2-a]pyridine-2-carboxylate in a mixture of 3.64 mL of tetrahydrofuran and 0.16 mL of methanol. The reaction mixture is stirred for 3 hours at 25° C. and then treated dropwise at 0° C. with 2 N hydrochloric acid HCl until a pH of 4 is reached. The precipitate formed after 20 minutes is filtered off by suction and washed with diethyl ether, and then dried under reduced pressure at 48° C. to give 280 mg of 6-pyridin-2-ylimidazo[1,2-a]pyridine-2-carboxylic acid in the form of a pasty pink solid.

¹H NMR spectrum (DMSO-d6, δ in ppm): 7.47 (m, 1H), 7.83 (d, J=9.8 Hz, 1H), 7.99 (dt, J=8.5, 2.0 Hz, 1H), 8.06 (d, J=8.5 Hz, 1H), 8.31 (broad d, J=9.8 Hz, 1H), 8.73 (m, 2H), 9.52 (broad s, 1H).

### 4. N-methoxy-N-methyl-6-pyridin-2-ylimidazo[1,2-a]pyridine-2-carboxamide

N-Methoxy-N-methyl-6-pyridin-2-ylimidazo[1,2-a]pyridine-2-carboxamide is prepared from 6-pyridin-2-ylimidazo[1,2-a]pyridine-2-carboxylic acid under conditions similar to those described for the preparation of intermediate 1.

¹H NMR spectrum (DMSO-d6, δ in ppm): 3.45 (broad s, 3H), 3.78 (s, 3H), 7.41 (ddd, J=7.6, 5.4, 1.2 Hz, 1H), 7.71 (d, J=9.3 Hz, 1H), 7.94 (td, J=7.6, 2.0 Hz, 1H), 8.00 (broad d, J=7.6 Hz, 1H), 8.05 (dd, J=9.3, 2.0 Hz, 1H), 8.53 (s, 1H), 8.70 (broad d, J=5.4 Hz, 1H), 9.38 (broad s, 1H).

Mass spectrum (LC-MS-DAD-ELSD): m/z 283 [M+H]⁺.

The tables that follow illustrate the chemical structures (Table 1) and the spectroscopic characteristics (Table 2) of a number of examples of compounds according to the invention.

In these tables, the compounds described are in the form of the base; “Me” means a methyl group.

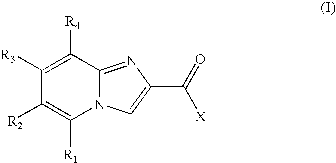

TABLE 1

| Ex | R₁ | R₂ | R₃ | R₄ | X |
|---|---|---|---|---|---|
| 1 | H | Cl | H | H | 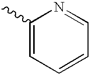 |
| 2 | H | Cl | H | H | 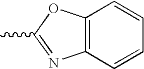 |
| 3 | H | Cl | H | H | 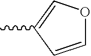 |
| 4 | H | Cl | H | H | 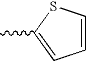 |
| 5 | H | Cl | H | H | 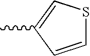 |
| 6 | H | Cl | H | H | 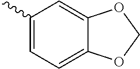 |
| 7 | H | Cl | H | H | 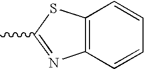 |
| 8 | H | Me | H | H | 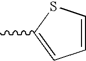 |
| 9 | Me | H | H | H | 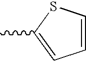 |
| 10 | H | 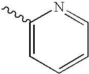 | H | H | 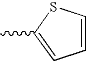 |

TABLE 2

| Ex. | Characterizations |
|---|---|
| 1 | ¹H NMR spectrum (DMSO-d6, δ in ppm): 7.42 (dd, J = 2.0 and 10.0 Hz, 1H), from 7.69 to |
| | 7.74 (m, 2H), 8.08 (broad t, J = 8.0 Hz, 1H), 8.13 (broad d, J = 8.0 Hz, 1H), 8.80 (broad d, J = |
| | 5.0 Hz, 1H), 8.95 (d, J = 2.0 Hz, 1H), 9.08 (s, 1H). |
| | Mass spectrum (LC-MS-DAD-ELSD): m/z 258 [M + H]⁺, presence of 1 Cl |
| 2 | ¹H NMR spectrum (DMSO-d6, δ in ppm): 7.48 (dd, J = 2.0 and 9.5 Hz, 1H), 7.59 (dt, J = 1.0 |
| | and 7.5 Hz, 1H), 7.68 (dt, J = 1.0 and 7.5 Hz, 1H), 7.77 (broad d, J = 9.5 Hz, 1H), 7.97 (broad |
| | d, J = 7.5 Hz, 1H), 8.04 (broad d, J = 7.5 Hz, 1H), 9.08 (dd, J = 1.0 and 2.0 Hz, 1H), 9.37 (d, J = |
| | 1.0 Hz, 1H). |
| | Mass spectrum (CI): m/z 298 [M + H]⁺, presence of 1 Cl |
| 3 | ¹H NMR spectrum (DMSO-d6, δ in ppm): 7.03 (broad d, J = 2.0 Hz, 1H), 7.44 (dd, J = 2.0 and |
| | 9.5 Hz, 1H), 7.77 (broad d, J = 9.5 Hz, 1H), 7.87 (t, J = 2.0 Hz, 1H), 8.57 (broad s, 1H), 8.89 |
| | (broad d, J = 2.0 Hz, 1H), 9.09 (broad d, J = 2.0 Hz, 1H). |
| | Mass spectrum (CI): m/z 247 [M + H]⁺, presence of 1 Cl |
| 4 | ¹H NMR spectrum (DMSO-d6, δ in ppm): 7.33 (dd, J = 4.0 and 5.0 Hz, 1H), 7.47 (dd, J = 2.0 |
| | and 9.5 Hz, 1H), 7.79 (broad d, J = 9.5 Hz, 1H), 8.11 (dd, J = 1.5 and 5.0 Hz, 1H), 8.62 (d, J = |
| | 1.0 Hz, 1H), 8.70 (dd, J = 1.5 and 4.0 Hz, 1H), 8.90 (dd, J = 1.0 and 2.0 Hz, 1H). |
| | Mass spectrum (CI): m/z 263 [M + H]⁺, presence of 1 Cl |
| 5 | ¹H NMR spectrum (DMSO-d6, δ in ppm): 7.44 (dd, J = 2.0 and 9.5 Hz, 1H), 7.68 (dd, J = 3.0 |
| | and 5.0 Hz, 1H), 7.78 (broad d, J = 9.5 Hz, 1H), 7.85 (dd, J = 1.5 and 5.0 Hz, 1H), 8.59 (d, J = |
| | 1.0 Hz, 1H), 8.90 (dd, J = 1.0 and 2.0 Hz, 1H), 9.19 (dd, J = 1.5 and 3.0 Hz, 1H). |
| | Mass spectrum (CI): m/z 263 [M + H]⁺, presence of 1 Cl |
| 6 | ¹H NMR spectrum (DMSO-d6, δ in ppm): 6.17 (s, 2H), 7.11 (d, J = 8.5 Hz, 1H), 7.43 (dd, J = |
| | 2.0 and 9.5 Hz, 1H), 7.79 (broad d, J = 9.5 Hz, 1H), 7.88 (d, J = 2.0 Hz, 1H), 8.19 (dd, J = 2.0 |
| | and 8.5 Hz, 1H), 8.54 (d, J = 1.0 Hz, 1H), 8.89 (dd, J = 1.0 and 2.0 Hz, 1H). |
| | Mass spectrum (CI): m/z 301 [M + H]⁺, presence of 1 Cl |
| 7 | ¹H NMR spectrum (DMSO-d6, δ in ppm): 7.48 (dd, J = 2.0 and 9.5 Hz, 1H), from 7.63 to 7.76 |
| | (m, 2H), 7.78 (broad d, J = 9.5 Hz, 1H), 8.30 (m, 2H), 9.06 (dd, J = 1.0 and 2.0 Hz, 1H), 9.39 |
| | (d, J = 1.0 Hz, 1H). |
| | Mass spectrum (CI): m/z 314 [M + H]⁺, presence of 1 Cl |
| 8 | ¹H NMR spectrum (DMSO-d6, δ in ppm): 2.31 (s, 3 H), 7.27 (dd, J = 9.3, 1.5 Hz, 1 H), 7.31 |
| | (dd, J = 4.0, 4.9 Hz, 1 H), 7.64 (d, J = 9.3 Hz, 1 H), 8.07 (dd, J = 4.9, 1.0 Hz, 1 H), 8.41 (broad d, |
| | J = 1.5 Hz, 1 H), 8.58 (s, 1 H), 8.70 (dd, J = 4.0, 1.0 Hz, 1 H) |
| | Mass spectrum (LC-MS-DAD-ELSD): m/z 243 [M + H]⁺. |
| 9 | ¹H NMR spectrum (DMSO-d6, δ in ppm): 2.68 (s, 3 H), 6.91 (d, J = 6.6 Hz, 1 H), 7.33 (dd, |
| | J = 5.1, 4.1 Hz, 1 H), 7.37 (dd, J = 9.1, 6.6 Hz, 1 H), 7.63 (d, J = 9.1 Hz, 1 H), 8.09 (dd, J = 5.1, 1.5 |
| | Hz, 1 H), 8.53 (s, 1 H), 8.73 (dd, J = 4.1, 1.5 Hz, 1 H) |
| | Mass spectrum (LC-MS-DAD-ELSD): m/z 243 [M + H]⁺. |
| 10 | ¹H NMR spectrum (DMSO-d6, δ in ppm): 7.34 (dd, J = 5.0, 3.8 Hz, 1H), 7.43 (ddd, J = 7.6, 4.7, |
| | 1.1 Hz, 1H), 7.84 (d, J = 9.5 Hz, 1H), 7.96 (td, J = 7.6, 2.0 Hz, 1H), 8.03 (broad d, J = 7.6 Hz, 1H), |
| | 8.08-8.16 (m, 2H), 8.69-8.75 (m, 2H), 8.77 (s, 1H), 9.42 (broad s, 1H). |
| | Mass spectrum (LC-MS-DAD-ELSD): m/z 306 [M + H]⁺. |

The compounds according to the invention underwent pharmacological tests to determine their modulatory effect on NOT.

Evaluation of the In Vitro Activity on N2A Cells

The activity of the compounds according to the invention was evaluated on a cell line (N2A) endogenously expressing the murine Nurr1 receptor and stably transfected with the NOT binding response element (NBRE) coupled to the luciferase reporter gene. The EC₅₀ values are between 0.01 and 1000 nM. The tests were performed according to the procedure described hereinbelow.

The cell line Neuro-2A is obtained from a standard commercial source (ATCC). The clone Neuro-2A was obtained from a spontaneous tumour originating from a strain of albino mice A by R. J Klebe et al. This line Neuro-2A is then stably transfected with 8NBRE-luciferase. The N2A-8NBRE cells are cultured to the point of confluence in 75 cm² culture flasks containing DMEM supplemented with 10% foetal calf serum, 4.5 g/L of glucose and 0.4 mg/ml of geneticin. After culturing for one week, the cells are recovered with 0.25% trypsin for 30 seconds and then resuspended in DMEM without phenol red, containing 4.5 g/L of glucose and 10% Hyclone defatted serum, and placed in white, transparent-based 96-well plates. The cells are deposited at a rate of 60 000 per well in 75 μL for 24 hours before adding the products. The products are applied in 25 μL and incubated for a further 24 hours. On the day of measurement, an equivalent volume (100 μL) of Steadylite is added to each well, and the wells are then left for 30 minutes to obtain complete lysis of the cells and maximum production of the signal. The plates are then measured in a microplate luminescence counter, after having been sealed with an adhesive film. The products are prepared in the form of a 10⁻² M stock solution, and then diluted in 100% of DMSO. Each concentration of product is prediluted in culture medium before incubation with the cells thus containing 0.625% final of DMSO.

For example, compound 4 gave an EC₅₀ value of 4.7 nM.

It is thus seen that the compounds according to the invention have a modulatory effect on NOT.

The compounds chosen from the compounds of formula (I) as defined above, and also 5-bromo-2-benzofuranyl)imidazo[1,2-a]pyridin-2-ylmethanone and (5-methoxy-2-benzofuranyl)-imidazo[1,2-a]pyridin-2-ylmethanone, and the addition salts of these compounds with a pharmaceutically acceptable acid, may thus be used for the preparation of medicaments for their therapeutic application in the treatment or prevention of diseases involving the NOT receptors.

Thus, according to another of its aspects, a subject of the invention is a medicament that comprises a compound chosen from the compounds of formula (I) as defined above, and also 5-bromo-2-benzofuranyl)imidazo[1,2-a]pyridin-2-ylmethanone and (5-methoxy-2-benzofuranyl)imidazo[1,2-a]pyridin-2-ylmethanone, and the addition salts of these compounds with a pharmaceutically acceptable acid, and more particularly that comprises a compound of formula (I) or an addition salt thereof with a pharmaceutically acceptable acid.

These medicaments find their therapeutic use especially in the treatment and prevention of neurodegenerative diseases, for instance Parkinson's disease, Alzheimer's disease, tauopathies (e.g. progressive supranuclear palsy, frontotemporal dementia, corticobasal degeneration, Pick's disease); cerebral trauma, for instance ischaemia and cranial trauma and epilepsy; psychiatric diseases, for instance schizophrenia, depression, substance dependency, and attention-deficit hyperactivity disorder; inflammatory diseases of the central nervous system, for instance multiple sclerosis, encephalitis, myelitis and encephalomyelitis and other inflammatory diseases, for instance vascular pathologies, atherosclerosis, joint inflammations, arthrosis, rheumatoid arthritis; osteoarthritis, Crohn's disease, ulcerative colitis; allergic inflammatory diseases such as asthma, autoimmune diseases, for instance type 1 diabetes, lupus, scleroderma, Guillain-Barré syndrome, Addison's disease and other immune-mediated diseases; osteoporosis; cancers.

Thus, the present invention is directed towards a compound chosen from the compounds of formula (I) as defined above, 5-bromo-2-benzofuranyl)imidazo[1,2-a]pyridin-2-ylmethanone and (5-methoxy-2-benzofuranyl)imidazo[1,2-a]pyridin-2-ylmethanone, and the addition salts of these compounds with a pharmaceutically acceptable acid, for the treatment or prevention of one of the abovementioned diseases.

According to one particular embodiment, these medicaments find their use in the treatment or prevention of one of the abovementioned diseases, with the exception of cancers.

According to another particular embodiment, these medicaments find their use in the treatment or prevention of one of the abovementioned diseases, with the exception of inflammatory diseases.

According to another of its aspects, the present invention relates to the use of a compound chosen from the compounds mentioned above, for the preparation of a medicament intended for the treatment or prevention of one of the abovementioned diseases.

These compounds may also be used as a treatment combined with grafts and/or transplantations of stem cells.

According to another of its aspects, the present invention relates to pharmaceutical compositions comprising, as active principle, a compound chosen from the group of compounds defined above. These pharmaceutical compositions contain an effective dose of at least one compound chosen from the group of compounds defined above, or a pharmaceutically acceptable salt of the said compound, and also at least one pharmaceutically acceptable excipient.

The said excipients are chosen, according to the pharmaceutical form and the desired mode of administration, from the usual excipients known to those skilled in the art.

In the pharmaceutical compositions of the present invention for oral, sublingual, subcutaneous, intramuscular, intravenous, topical, local, intratracheal, intranasal, transdermal or rectal administration, the active principle chosen from the group of compounds defined above, or the salt thereof, may be administered in unit administration form, as a mixture with standard pharmaceutical excipients, to man and animals for the prophylaxis or treatment of the above complaints or diseases.

The appropriate unit forms of administration include oral forms such as tablets, soft or hard gel capsules, powders, granules and oral solutions or suspensions, sublingual, buccal, intratracheal, intraocular, intranasal or inhalation administration forms, topical, transdermal, subcutaneous, intramuscular or intravenous administration forms, rectal administration forms and implants. For topical application, the compounds according to the invention may be used in creams, gels, ointments or lotions.

By way of example, a unit administration form of a compound according to the invention in tablet form may comprise the following components:

| | |
|---|---|
| Compound according to the invention | 50.0 mg |
| Mannitol | 223.75 mg |
| Croscarmellose sodium | 6.0 mg |
| Corn starch | 15.0 mg |
| Hydroxypropylmethylcellulose | 2.25 mg |
| Magnesium stearate | 3.0 mg |

There may be particular cases in which higher or lower dosages are appropriate; such dosages are not outside the context of the invention. According to the usual practice, the dosage that is appropriate for each patient is determined by the doctor according to the mode of administration and the weight and response of the said patient.

According to another of its aspects, the present invention also relates to a method for treating the pathologies indicated above, which comprises the administration, to a patient, of an effective dose of a compound chosen from the group of compounds defined above, or a pharmaceutically acceptable salt thereof.

It is understood that all the subjects defined above, especially the medicament, pharmaceutical composition and treatment method, also apply more particularly to the subgroups of compounds previously defined.

What is claimed is:

1. A compounds of formula (I): 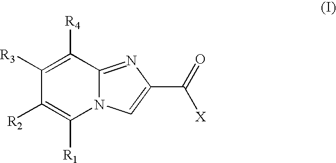
wherein:
X represents a benzodioxole group, or a heteroaromatic group linked to the rest of the molecule via a carbon atom, this group being optionally substituted with one or more groups chosen, independently of each other, from a halogen atom and a group (C₁-C₆)alkyl, (C₁-C₆)alkoxy or NRaRb;
R₂ represents
a hydrogen atom,
a halogen atom,
a group (C₁-C₆)alkyl optionally substituted with one or more atoms or groups chosen,
independently of each other, from halogen, hydroxyl and NRaRb,
a group (C₁-C₆)alkoxy optionally substituted with one or more atoms or groups chosen,
independently of each other, from halogen, hydroxyl and NRaRb,
a group (C₁-C₆)alkylthio,
a group (C₂-C₆)alkenyl,
a group (C₂-C₆)alkynyl,
a group —CO—R₅,
a group —CO—NR₆R₇,
a group —CO—O—R₈,
a group NR₉—CO—R₁₀,
a group —NR₁₁R₁₂,
a cyano group,
a phenyl group optionally substituted with one or more groups chosen, independently of each other, from halogen, (C₁-C₆)alkoxy, (C₁-C₆)alkyl optionally substituted with one or more atoms or groups: hydroxyl, NRcRd, CO—R₅, —CO—NR₆R₇, —CO—O—R₈, halogen or cyano, or
a heterocyclic group optionally substituted with one or more groups chosen, independently of each other, from the following atoms or groups: hydroxyl, halogen, (C₁-C₆)alkoxy, (C₁-C₆)alkyl optionally substituted with one or more hydroxyl, NRcRd, —CO—R₅, —CO—NR₆R₇, —CO—O—R₈, —NR₉—CO—R₁₀, cyano, and an oxido group;
R₁ represents a hydrogen atom, a halogen atom, a group (C₁-C₆)alkyl, a group (C₁-C₆)alkoxy or a hydroxyl or amino group; the group (C₁-C₆)alkyl possibly being substituted with one or more of the atoms or groups: halogens, hydroxyl; amino, and (C₁-C₆)alkoxy, and the group (C₁-C₆)alkoxy possibly being substituted with one or more of the atoms or groups: halogen, hydroxyl, amino, or (C₁-C₆)alkoxy;
R₃ represents a hydrogen atom, a halogen atom or a group (C₁-C₆)alkyl or hydroxyl;
R₄ represents a hydrogen atom or a halogen atom;
R₅ represents a hydrogen atom or a group (C₁-C₆)alkyl;
R₆ and R₇, which may be identical or different, represent a hydrogen atom or a group (C₁-C₆)alkyl or form, with the nitrogen atom that bears them, a 4- to 7-membered ring optionally including another heteroatom chosen from N, O and S;
R₈ represents a group (C₁-C₆)alkyl;
R₉ and R₁₀, which may be identical or different, represent a hydrogen atom or a group (C₁-C₆)alkyl;
R₁₁ represents a group (C₁-C₆)alkyl;
R₁₂ represents a hydrogen atom or a group (C₁-C₆)alkyl;
Ra and Rb represent, independently of each other, a hydrogen atom, a group (C₁-C₆)alkyl or form, with the nitrogen atom that bears them, a 4- to 7-membered ring, optionally including another heteroatom chosen from N, O and S; and
Rc and Rd represent a hydrogen atom or a (C₁-C₆)alkyl;
or an acid addition salt thereof;
with the exception of (5-bromo-2-benzofuranyl)imidazo[1,2-a]pyridin-2-ylmethanone and (5-methoxy-2-benzofuranyl)imidazo[1,2-a]pyridin-2-ylmethanone.

2. The compound of formula (I) according to claim 1, wherein at least one from among R₁, R₂, R₃ and R₄ is other than a hydrogen atom, the other groups being as defined in claim 1; or an acid addition salt thereof.

3. The compound of formula (I) according to claim 1, wherein R₂ represents one of the following groups:
a hydrogen atom,
a halogen atom,
a group (C₁-C₆)alkyl, or
a monocyclic heterocyclic group of 5 or 6 atoms, optionally substituted with one or more groups chosen, independently of each other, from the following atoms or groups: hydroxyl, halogen, (C₁-C₆)alkoxy, (C₁-C₆)alkyl optionally substituted with one or more hydroxyl, NRcRd, —CO—R₅, —CO—NR₆R₇, —CO—O—R₈, —NR₉—CO—R₁₀, cyano, and an oxido group;
R₅ represents a hydrogen atom or a group (C₁-C₆)alkyl; and
Rc and Rd represent a hydrogen atom;
or an acid addition salt thereof.

4. The compound of formula (I) according to claim 1, wherein X represents a pyridine, benzoxazole, thiophene, furan, benzodioxole or benzothiazole group;
or an acid addition salt thereof.

5. The compound of formula (I) according to claim 1, wherein:
R₁ represents a hydrogen atom or a methyl group;
R₃ and R₄ represent a hydrogen atom;
R₂ represents a hydrogen atom, a chlorine atom, a methyl group or a pyridine group;
X represents a pyridine, benzoxazole, thiophene, furan, benzodioxole or benzothiazole group, and at least one from among R₁, R₂, R₃ and R₄ is not hydrogen,
or an acid addition salt thereof.

6. The compound of formula (I) according to claim 1, selected from the group consisting of:
(6-Chloroimidazo[1,2-a]pyridin-2-yl)(pyridin-2-yl)methanone;
Benzoxazol-2-yl(6-chloroimidazo[1,2-a]pyridin-2-yl)methanone;
(6-Chloroimidazo[1,2-a]pyridin-2-yl)(3-furyl)methanone;
(6-Chloroimidazo[1,2-a]pyridin-2-yl)(thien-2-yl)methanone;
(6-Chloroimidazo[1,2-a]pyridin-2-yl)(thien-3-yl)methanone;
1,3-Benzodioxol-5-yl(6-chloroimidazo[1,2-a]pyridin-2-yl)methanone;
Benzothiazol-2-yl(6-chloroimidazo[1,2-a]pyridin-2-yl)methanone;
(6-Methylimidazo[1,2-a]pyridin-2-yl)(thien-2-yl)methanone;
(5-Methylimidazo[1,2-a]pyridin-2-yl)(thien-2-yl)methanone; and
(6-Pyridin-2-yl)imidazo[1,2-a]pyridin-2-yl)(thien-2-yl)methanone;
or an addition salt thereof with a pharmaceutically acceptable acid.

7. A pharmaceutical composition comprising a compound selected from a compound according to claim 1, 5-bromo-2-benzofuranyl)imidazo[1,2-a]pyridin-2-ylmethanone and (5-methoxy-2-benzofuranyl)imidazo[1,2-a]pyridin-2-ylmethanone, or a pharmaceutically acceptable salt thereof; and also at least one pharmaceutically acceptable excipient.

8. A pharmaceutical composition comprising a compound according to claim 1, or a pharmaceutically acceptable salt thereof; and also at least one pharmaceutically acceptable excipient.

9. A method for treating or preventing a disease comprising administering to a patient an effective amount of a compound selected from the group consisting of a compound according to claim 1, 5-bromo-2-benzofuranyl)imidazo[1,2-a]pyridin-2-ylmethanone and (5-methoxy-2-benzofuranyl)imidazo[1,2-a]pyridin-2-ylmethanone, or a pharmaceutically acceptable salt thereof.

10. The method according to claim 9 wherein the disease is a neurodegenerative disease.

11. The method according to claim 9 wherein the disease is a cerebral trauma or epilepsy.

12. The method according to claim 9 wherein the disease is a psychiatric disease.

13. The method according to claim 9 wherein the disease is an inflammatory disease.

14. The method according to claim 9 wherein the disease is a osteoporosis.

15. The method according to claim 9 wherein the disease is cancer.

16. The method according to claim 9 wherein the disease is Parkinson's disease, Alzheimer's disease, tauopathies or multiple sclerosis.

17. The method according to claim 9 wherein the disease is schizophrenia, depression, substance dependency or attention-deficit hyperactivity disorder.

18. A compound selected from the group consisting of:
N-methoxy-N-methyl-6-methylimidazo[1,2-a]pyridine-2-carboxamide;
ethyl 6-(4,4,5,5-tetramethyl-1,3,2-dioxaborolan-2-yl)imidazo[1,2-a]pyridine-2-carboxylate hydrobromide;
ethyl 6-pyridin-2-ylimidazo[1,2-a]pyridine-2-carboxylate;
6-pyridin-2-ylimidazo[1,2-a]pyridine-2-carboxylic acid; and
N-methoxy-N-methyl-6-pyridin-2-ylimidazo[1,2-a]pyridine-2-carboxamide.
